(19) United States
(12) Patent Application Publication
(10) Pub. No.: US 2025/0312156 A1
(43) Pub. Date: Oct. 9, 2025

(54) TRANSCATHETER SYSTEMS AND METHODS FOR ATRAUMATIC DELIVERY AND INSTALLATION OF A TISSUE ANCHOR IN A HEART OF A SUBJECT

(71) Applicant: Edwards Lifesciences Innovation (Israel) Ltd., Caesarea (IL)

(72) Inventors: Eran Hoffer, Yahud (IL); Aviv Galon, Tel Aviv (IL); Ido Halabi, Givatayim (IL); Ofry Efraim Yossef, Tel-Aviv (IL); Haim Brauon, Beit Dagan (IL); Or Cohen, Modi'in (IL); Yaron Herman, Givat Ada (IL)

(21) Appl. No.: 19/240,474

(22) Filed: Jun. 17, 2025

Related U.S. Application Data
(63) Continuation of application No. PCT/IB2023/062843, filed on Dec. 17, 2023.
(60) Provisional application No. 63/476,125, filed on Dec. 19, 2022.

(51) Int. Cl. A61F 2/24

(52) U.S. Cl. CPC A61F 2/2466; A61F 2220/0016

(57) ABSTRACT

A tissue anchor includes a sharp tissue-engaging element and a driving interface which is fixed with respect to the tissue-engaging element. A tube has a blunt cap reversibly attached to and covering a distal end of the tube, and is transluminally advanceable in a distal direction toward an anatomical site within a subject. A driver, while engaged with the driving interface of the tissue anchor, is configured to drive the tissue-engaging element through the blunt cap and into tissue at the anatomical site, such that the blunt cap becomes an anchor-head of the tissue anchor, secured to the tissue by the tissue-engaging element. Other embodiments are also described.

### CROSS-REFERENCE TO RELATED APPLICATIONS

This application is a continuation of International Patent Application No. PCT/IB2023/062843, filed Dec. 17, 2023, which claims the benefit of U.S. Patent Application No. 63/476,125, filed Dec. 19, 2022, the entire disclosures all of which are incorporated by reference for all purposes.

### BACKGROUND

The demand for transcatheter cardiac surgeries and medical processes in mammalian, and specifically human, hearts, has been increasing. Such procedures may necessitate insertion of a tissue anchor into the cardiac tissue. If a tissue anchor has a sharp tip and is advanced transvascularly, there is a risk that the sharp tip of the tissue anchor could cause damage, such as nicks or punctures to blood vessels.

There is thus a need for techniques and devices for atraumatic introduction of a tissue anchor, while ensuring that a sharp tip of the tissue anchor does not cause damage to blood vessels, the heart, or other anatomy during the introduction.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

In accordance with some implementations herein, a tissue anchor is delivered to a tissue inside a body (e.g., tissue in a heart chamber) in a safe manner, enclosed within a tube having an atraumatic, or blunt, cap.

In some implementations, the tissue anchor is delivered to the tissue (e.g., to tissue of a heart chamber) within the tube, where the tube has a blunt cap.

In some implementations, the tissue anchor is advanced into the tissue through the blunt cap, such that the blunt cap is pushed against the tissue and becomes an anchor head.

In some implementations, the blunt cap includes multiple segments, which are adapted to move relative to the tube, to form an opening for the tissue anchor to pass through.

In accordance with some implementations, there is provided a system for use with a subject, the system including a tissue anchor, including a tissue-engaging element and a driving interface which is fixed with respect to the tissue-engaging element.

The system can further include a tube, having a blunt cap closing a distal end of the tube. The tube is configured to be transluminally advanced toward an anatomical site of the subject, with the tissue anchor disposed within the tube, proximally from the blunt cap.

The system can further include a driver, extending through the tube. The driver is configured to engage the driving interface of the tissue anchor, advance the tissue-engaging element distally beyond the cap. The driver is further configured to anchor the tissue anchor at the anatomical site by driving the tissue-engaging element into tissue at the anatomical site.

In some implementations, the tube can include a distal end of a longitudinal catheter.

In some implementations, the system can further include a longitudinal catheter. The tube can include a sheath attached to a distal end of the longitudinal catheter.

In some implementations, the blunt cap can be integrally formed with the tube.

In some implementations, the blunt cap can be attached to the distal end of the tube.

In some implementations, the tissue-engaging element of the tissue anchor can be adapted to be driven through the blunt cap of the tube and into the tissue. In some implementations, the anchoring of the tissue anchor to the tissue secures the blunt cap against the tissue. In some implementations, this can facilitate detachment of the blunt cap from the body of the tube and convert the blunt cap into an anchor-head of the tissue anchor.

In some implementations, the blunt cap can include a predefined opening for passage of the tissue-engaging element of the tissue anchor through the blunt cap.

In some implementations, the blunt cap may be pierceable by the tissue-engaging element during driving of the tissue-engaging element through the blunt cap of the tube.

In some implementations, the tissue-engaging element can include a helical tissue-engaging element, adapted to be helically driven through the blunt cap.

In some implementations, the tissue-engaging element can include a linear tissue-engaging element, adapted to be driven longitudinally through the blunt cap. In some implementations, the linear tissue-engaging element can include a dart or a dowel.

In some implementations, the blunt cap can be attached to the body of the tube body by a snap fit engagement that has a predefined strength.

In some implementations, following driving of the tissue-engaging element into the tissue, the longitudinal catheter is adapted to test anchoring of the tissue-engaging element to the tissue by drawing the tube proximally away from the tissue. Only if the tissue-engaging element is sufficiently anchored to the tissue, the drawing of the tube proximally away from the tissue overcomes the predefined strength of the snap-fit to detach the blunt cap from the body of the tube.

In some implementations, the blunt cap can include a plurality of segments forming a tapered structure. During anchoring of the tissue anchor, motion of the segments outwardly in a radial direction is triggered, exposing the tissue-engaging element of the tissue anchor for anchoring thereof.

In some implementations, each of the segments can include a first portion and second portion. The second portion can be pivotable relative to the first portion. The second portions of the segments can be adapted such that pressure applied to the second portions by the tissue causes said second portions move proximally and outwardly in the radial direction.

In some implementations, the system can further include a biasing element connecting the first portion of each of the segments to the tube and biasing the second portion away from the distal end of the tube. The pressure applied by the tissue can cause the biasing element to retract, allowing the second portions to move proximally toward the end of the tube.

In some implementations, the distal portion of the tube has a smaller diameter than a proximal portion of the tube. A shoulder is formed between the distal portion and the proximal portion on an external side of the tube, and the shoulder functions as a seat for the biasing element.

In some implementations, each of the segments is pivotably attached to the distal end of the tube, and is pivotable relative thereto. During anchoring, the driver is adapted to advance the tissue anchor distally relative to the segments, pushing the segments to pivot laterally away from each other in the radial direction. This can form an opening for passage of the tissue anchor.

In some implementations, the tissue anchor includes a head portion having a sloped surface. Each of the segments is adapted, during advancing of the tissue anchor, to slide along the sloped surface. This causes pivoting of the segments outwardly in the radial direction.

In some implementations, for each of the segments, an arm is fixed to the segment to form a structure that is (i) pivotably coupled to the distal end of the tube, and (ii) biased to pivot such that the arm deflects medially and the segment deflects laterally.

In some implementations, in a delivery state of the system, a head portion of the tissue anchor can be disposed between the arms in a manner that obstructs the arms from deflecting medially, thereby obstructing the structures from pivoting, and thereby holding the segments medially to form the blunt cap.

In some implementations, advancement of the tissue anchor distally away from the arms allows the structures to pivot such that the segments pivot outwardly in the radial direction and form an opening for passage of the tissue anchor.

In accordance with some implementations, there is provided a system for use with a subject, the system including a tissue anchor, including a tissue-engaging element and a driving interface which is fixed with respect to the tissue-engaging element.

In some implementations, the system can further include a tube, having a blunt cap reversibly attached to and closing a distal end of the tube. In some implementations, the tube is configured to be transluminally advanced toward an anatomical site of the subject with the tissue anchor disposed within the tube, proximally from the blunt cap.

In some implementations, the system can further include a driver, extending through the tube. In some implementations, the driver can be configured to anchor the tissue anchor at the anatomical site by driving the tissue-engaging element into tissue at the anatomical site.

In some implementations, the tissue-engaging element of the tissue anchor is adapted to be driven through the blunt cap of the tube and into the tissue. As such, anchoring of the tissue anchor to the tissue secures the blunt cap against the tissue. In some implementations, this may facilitate detachment of the blunt cap from the body of the tube, and convert the blunt cap into an anchor-head of the tissue anchor.

In some implementations, the tube can include a distal end of a longitudinal catheter.

In some implementations, the system can further include a longitudinal catheter. The tube can include a sheath attached to a distal end of the longitudinal catheter.

In some implementations, the blunt cap can include a predefined opening for passage of the tissue-engaging element of the tissue anchor through the blunt cap.

In some implementations, the blunt cap may be pierceable by the tissue-engaging element during driving of the tissue-engaging element through the blunt cap of the tube.

In some implementations, the tissue-engaging element can include a helical tissue-engaging element, adapted to be helically driven through the blunt cap.

In some implementations, the tissue-engaging element can include a linear tissue-engaging element, adapted to be driven longitudinally through the blunt cap. In some implementations, the linear tissue-engaging element can include a dart, a lance, or a dowel.

In some implementations, the blunt cap can be attached to the body of the tube body by snap fit engagement, having a predefined strength.

In some implementations, following driving of the tissue-engaging element into the tissue, the longitudinal catheter is adapted to test anchoring of the tissue-engaging element to the tissue by drawing the tube proximally away from the tissue.

In some implementations, the system is configured such that drawing of the tube proximally away from the tissue overcomes the predefined strength of the snap-fit and detaches the blunt cap from the body of the tube only if the tissue-engaging element is sufficiently anchored to the tissue.

In accordance with some implementations, there is provided a method for anchoring a tissue anchor having a tissue-engaging element in tissue at a real or simulated anatomical site of a subject. In some implementations, the method includes transluminally advancing a tube, having a blunt cap closing a distal end of the tube, toward the anatomical site, with the tissue anchor disposed within the tube, proximally from the blunt cap.

In some implementations, the method can further include, advancing the tissue-engaging element distally beyond the blunt cap.

In some implementations, the method can further include anchoring the tissue anchor at the anatomical site by driving the tissue-engaging element into tissue at the anatomical site.

In some implementations, the advancing of the tissue-engaging element distally beyond the blunt cap can include driving the tissue-engaging element through the blunt cap of the tube and into the tissue. In some implementations, the method can further include, following the anchoring of the tissue anchor, converting the blunt cap into an anchor-head of the tissue anchor by detaching the blunt cap from the body of the tube.

In some implementations, the blunt cap can include a predefined opening for passage of the tissue-engaging element of the tissue anchor. In some implementations, the driving of the tissue-engaging element through the blunt cap can include driving the tissue- engaging element through the predefined opening.

In some implementations, the driving of the tissue-engaging element through the blunt cap can include, using the tissue-engaging element, piercing a hole through the blunt cap, for the tissue-engaging element to extend distally beyond the blunt cap.

In some implementations, the tissue-engaging element can include a helical tissue-engaging element. In some implementations, the driving of the tissue-engaging element can include helically driving the tissue-engaging element through the blunt cap.

In some implementations, the tissue-engaging element can include a linear tissue-engaging element. In some implementations, the driving of the tissue-engaging element can include longitudinally driving the tissue-engaging element through the blunt cap.

In some implementations, the method can further include following driving of the tissue-engaging element into the tissue, testing anchoring of the tissue-engaging element to the tissue by drawing the tube proximally away from the tissue.

In some implementations, the system is configured such that drawing of the tube proximally away from the tissue overcomes a predefined strength to detach the blunt cap from the body of the tube only if the tissue-engaging element is sufficiently anchored to the tissue.

In some implementations, the blunt cap can include a plurality of segments forming a substantially tapered structure. In some implementations, the advancing of the tissue-engaging element distally beyond the blunt cap can include moving the segments outwardly in a radial direction to expose the tissue-engaging element of the tissue anchor for anchoring thereof.

In some implementations, the moving of the segments can include causing the segments to pivot outwardly in the radial direction.

In accordance with some implementations, there is provided method for anchoring a tissue anchor having a tissue-engaging element in tissue at an anatomical site of a subject, the method including transluminally advancing a tube, having a blunt cap reversibly attached to and closing a distal end of the tube, toward the anatomical site of the subject, with the tissue anchor disposed within the tube, proximally from the blunt cap.

In some implementations, the method can include anchoring the tissue anchor at the anatomical site by driving the tissue-engaging element, through the blunt cap of the tube and into tissue at the anatomical site.

In some implementations, the method can include, following the anchoring of the tissue anchor, converting the blunt cap into an anchor-head of the tissue anchor by detaching the blunt cap from the body of the tube.

In some implementations, the blunt cap can include a predefined opening for passage of the tissue-engaging element of the tissue anchor through the blunt cap. In some implementations, the driving of the tissue-engaging element through the blunt cap can include driving the tissue-engaging element through the predefined opening.

In some implementations, the driving of the tissue-engaging element through the blunt cap can include, using the tissue-engaging element, piercing a hole through the blunt cap, for the tissue-engaging element to extend distally beyond the blunt cap.

In some implementations, the tissue-engaging element can include a helical tissue-engaging element. In some implementations, the driving of the tissue-engaging element can include helically driving the tissue-engaging element through the blunt cap.

In some implementations, the tissue-engaging element can include a linear tissue-engaging element. In some implementations, the driving of the tissue-engaging element can include longitudinally driving the tissue-engaging element through the blunt cap.

In some implementations, the detaching can include testing anchoring of the tissue-engaging element to the tissue by drawing the tube proximally away from the tissue.

In some implementations, the drawing of the tube proximally away from the tissue overcomes a predefined strength to detach the blunt cap detaches from the body of the tube only if the tissue-engaging element is sufficiently anchored to the tissue.

Any of the above method(s) can be performed on a living subject (e.g., human or other animal) or on a simulation (e.g., a cadaver, cadaver heart, anthropomorphic ghost, simulator, etc.). With a simulation, the body parts can optionally be referred to as “simulated” (e.g., simulated heart, simulated tissue, etc.) and can comprise computerized or physical representations.

Any of the above systems, devices, apparatuses, components, etc. can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

The present invention will be more fully understood from the following detailed description of implementations thereof, taken together with the drawings.

### BRIEF DESCRIPTION OF THE DRAWINGS

The foregoing discussion will be understood more readily from the following detailed description when taken in conjunction with the accompanying Figures, in which:

FIGS. 1A and 1B are, respectively, a perspective view illustration and a sectional illustration of an example system for delivery and installation of a tissue anchor, in accordance with some implementations, in a delivery state;

FIGS. 1C and 1D, are, respectively, a perspective view illustration and a sectional illustration of an example tissue anchor of the system of FIGS. 1A and 1B, in an anchored state, in accordance with some implementations;

FIG. 2 is a schematic sectional illustration of an example system for delivery and installation of a tissue anchor, and of phases of use thereof, in accordance with some implementations;

FIG. 3 is a schematic sectional illustration of an example system for delivery and installation of a tissue anchor, and of phases of use thereof, in accordance with some implementations;

FIG. 4 is a schematic sectional illustration of an example system for delivery and installation of a tissue anchor, and of phases of use thereof, in accordance with some implementations; and

FIG. 5 is a schematic sectional illustration of an example system for delivery and installation of a tissue anchor, and of phases of use thereof, in accordance with some implementations.

### DETAILED DESCRIPTION

The principles of the devices and methods for delivery and installation of a tissue anchor in tissue of a heart of a subject, and specifically the devices and methods for transcatheter or transluminal safe delivery and installation of such tissue anchors, may be better understood with reference to the drawings and the following description.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features can be omitted or simplified in order not to obscure the disclosure. Additionally, in order to avoid undue clutter from having too many reference numbers and lead lines on a particular drawing, some elements may not be explicitly identified in every drawing that contains that element.

It is to be understood that the scope of the invention is not limited in its application to the details of construction and the arrangement of the components set forth in the following description or illustrated in the drawings. The invention is capable of other implementations or of being practiced or carried out in various ways. Furthermore, it is to be understood that the phraseology and terminology employed in the disclosure is for the purpose of description and should not be regarded as limiting.

For the purposes of this application, the term “subject” relates to any mammal, particularly humans.

For the purposes of this application, the term “cardiac tissue” relates to any tissue of the heart, and includes, for example, any wall of the heart and any tissue of any heart valve.

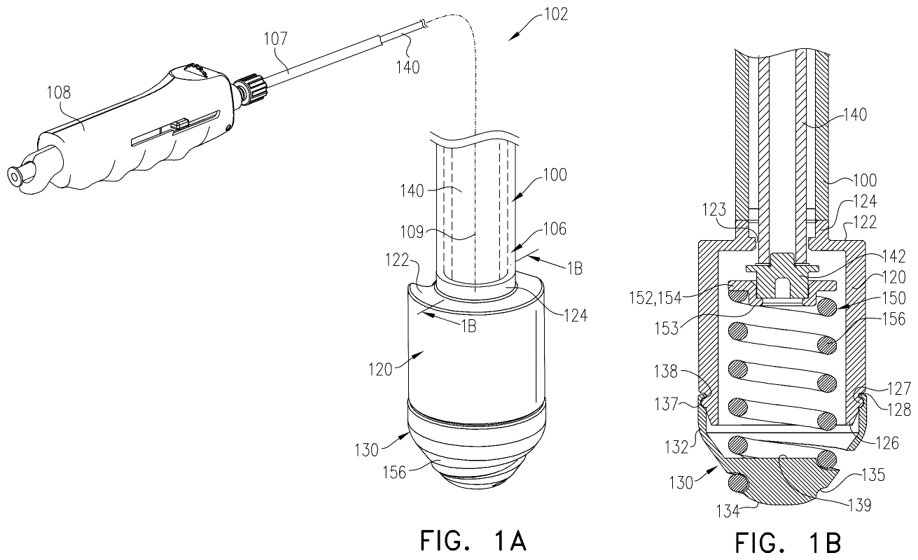

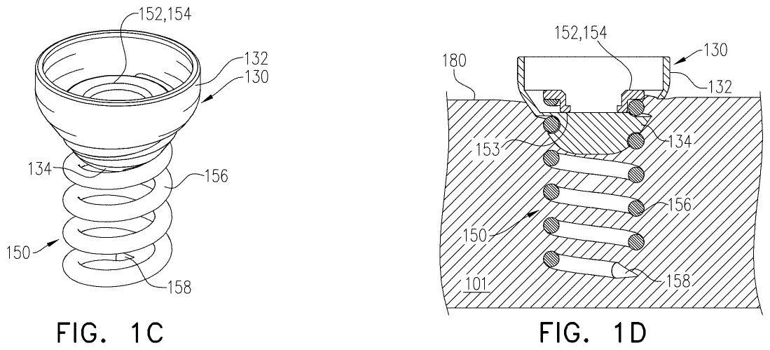

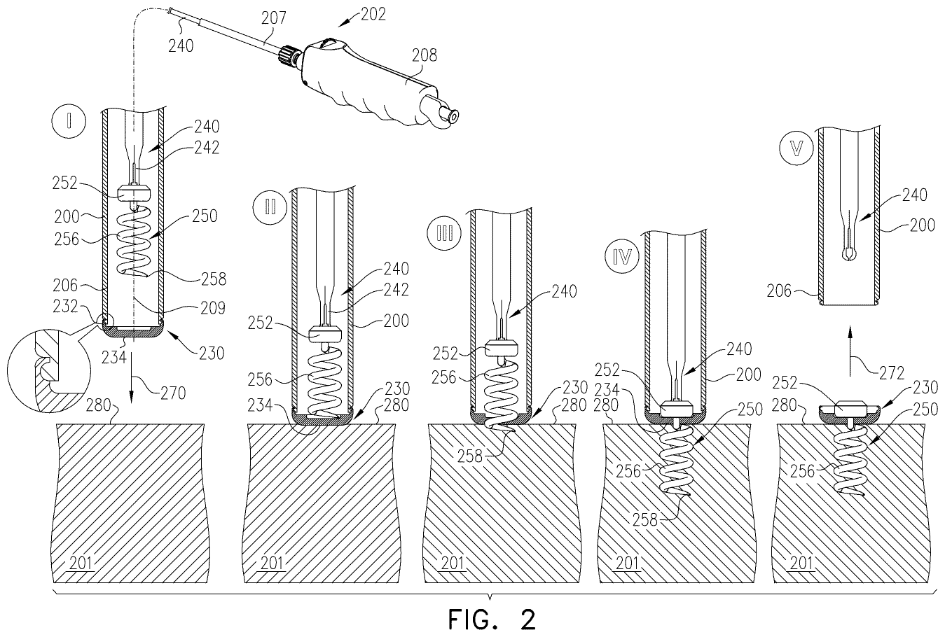

Reference is now made to FIGS. 1A-D, and FIG. 2, which illustrate a system in which a tissue anchor can be advanced intracorporeally within a tube that has an atraumatic cap, in accordance with some implementations. At the tissue, the tissue anchor is driven into the tissue in a manner that pins the cap against the tissue. The cap can be detached from the tube and left behind implanted at the tissue—e.g., serving as (e.g., becoming) the head of the anchor.

Referring now to the drawings, a system for delivery and installation of a tissue anchor according to some implementations is schematically shown in FIGS. 1A and 1B, in a delivery state, or mode of operation.

As seen in FIGS. 1A and 1B, in some implementations, a delivery tool 102 comprises a longitudinal catheter 100 that is adapted to be advanced toward an anatomical site. In some implementations, the anatomical site can be within the heart of the subject. For example, a distal part 106 of the catheter can be advanced to a chamber (e.g., a left atrium) of the heart (or to another location or anatomical site inside a body).

In some implementations, catheter 100 also has an extracorporeal proximal part 107 functionally associated with (e.g., coupled to) a control handle 108 of delivery tool 102. Distal part 106 of the catheter is guidable to the anatomical site, such as by being actively steerable (e.g., by distal part 106 being operatively coupled to one or more pullwires that extend from a steering controller in control handle 108), or by being passively guided and/or steered (e.g., by extending over or through another steerable element, such as an actively steerable catheter). In some implementations, a longitudinal axis 109 extends between proximal part 107 and steerable distal part 106 of catheter 100.

Catheter 100 can be adapted to be transluminally advanced to the anatomical site using any method known in the art, for example via the vena cava and, if necessary, via the septum separating the atria of the heart and/or via any other blood vessel of the body.

In some implementations, a tube 120 is attached to distal part 106 of catheter 100. In some implementations, catheter 100 and tube 120 are formed from a unitary tube (e.g., a single tube is molded and/or shaped to form both the catheter and the tube. Forming the catheter 100 and the tube 120 from a unitary tube can obviate the need to fixedly attach these two components to each other.

In other implementations, tube 120 comprises and/or is formed from a different piece of stock tubing than catheter 100, and these two components are welded and/or fixedly attached together.

In some implementations, tube 120 is in the form of a capsule, and includes a proximal annular wall 122 defining a portal 123, and a neck portion 124. In some implementations, distal part 106 of catheter 100 is fixedly attached to neck portion 124 and/or to annular wall 122. Tube 120 can terminate at a distal end 126.

In order to shield tissue from the distal end 126 of tube 120, a blunt cap 130 can be reversibly attached to the tube. In some implementations, blunt cap 130 defines a distal blunt end 134 that can be atraumatic—e.g., by being rounded and/or tapered.

In some implementations, blunt end 134 may define an external, substantially helical groove 135. In some implementations, blunt cap 130 has a generally tapered shape. In some implementations, proximal wall 132 of the blunt cap 130 is reversibly attached to distal end 126 of tube 120, for example by snap fit engagement.

In the illustrated example, distal end 126 of tube 120 includes a recess distal protrusion 127, and an indentation 128 proximally adjacent the distal protrusion. In some implementations, blunt cap 130 may cover (e.g., close), at least partially, the distal end of tube 120.

In some implementations, cap 130 covers (e.g., closes) the distal end completely. In some implementations, the cap does not cover (e.g., close) the distal end completely, but simply provides an atraumatic covering to the distal end of the tube/catheter. For example, the cap may only cover the rim of the tube, and/or may circumscribe an aperture through which tissue-engaging element 156 is advanceable.

In some implementations, the proximal end of proximal wall 132 of the blunt cap 130 includes a proximal indentation 137, adapted to receive protrusion 127, such that an end 138 of proximal wall 132 fits into indentation 128.

As explained in further detail hereinbelow, the snap fit engagement between tube 120 and blunt cap 130 can have a predefined strength (e.g., tolerance), which can be used to determine whether a tissue anchor has been properly installed.

In some implementations, delivery tool 102 further comprises a driver 140 that extends through catheter 100 and portal 123 of tube 120, into the hollow of tube 120. Driver 140 can be transluminally advanced through catheter 100 and can be controlled by handle 108.

In some implementations, driver 140 includes an anchor-engaging interface (e.g., a drive head) 142, adapted to engage a tissue anchor 150. As shown in FIG. 1B, driver 140 can include a longitudinal catheter extending through a lumen of catheter 100. In some implementations, and as shown in FIG. 1B, in the delivery state, driver 140 (e.g., anchor-engaging interface 142) can extend distally out of catheter 100 and into tube 120.

In some implementations, tissue anchor 150 includes a head portion 152 defining a driving interface 154, and a tissue-engaging element 156. In the example shown, tissue-engaging element is a helical tissue-engaging element—e.g., configured to be screwed into the tissue by application of torque to interface 154. However, the scope of the present disclosure includes other tissue-engaging elements such as (but not limited to) dart-like, or staple-like tissue-engaging elements. In some implementations, driving interface 154 is adapted to be reversibly engaged by anchor-engaging interface 142 of driver 140.

In some implementations, a proximal end of tissue-engaging element 156 is fixedly attached to head portion 152, and such that tissue-engaging element 156 is fixed with respect to the driving interface. Tissue-engaging element 156 can terminate in a sharp distal tip 158.

In some implementations, while the tissue anchor is delivered toward the tissue (e.g., in a delivery state of the system), and prior to driving of the tissue anchor into the tissue, a distal portion of tissue-engaging element 156, including distal tip 158, is arranged helically around distal blunt end 134 of blunt cap 130. As such, a distal portion of tissue-engaging element 156 can be disposed external to blunt cap 130, while a proximal portion of tissue-engaging element 156 is disposed within the blunt cap and within tube 120.

Alternatively, in some implementations, the entire tissue anchor 150 (e.g., including tissue-engaging element 156 and tip 158) is housed within tube 120, proximally from blunt cap 130, during delivery of the tissue anchor to the tissue, and/or during positioning of the tube at the tissue.

In some implementations, during anchoring of the tissue anchor, driver 140 advances tissue anchor 150 distally relative to tube 120 and to blunt cap 130, so that tissue-engaging element 156 extends farther distally beyond the blunt cap. At this stage, tube 120 remains attached to blunt cap 130, and head portion 152 of tissue anchor 150 remains within tube 120 or within blunt cap 130.

In some implementations, driver 140 (e.g., anchor-engaging interface 142 thereof) is rotatable to apply torque to driving interface 154 and screw tissue-engaging element 156 into tissue at the anatomical site.

In some implementations, tissue-engaging element 156 is driven through blunt cap 130 and into the tissue.

For example, blunt cap 130 can include a predefined opening for passage of tissue-engaging element 156 through the blunt cap. In such implementations, cap 130 may only partially cover the distal end of tube 120.

In some implementations, blunt cap 130 is pierceable by tissue-engaging element 156, such that the tissue-engaging element pierces through the blunt cap of the tube as it is driven distally.

In such implementations, cap 130 may completely cover (e.g., seal) the distal end of tube 120. In implementations in which the tissue-engaging element is a linear tissue-engaging element, the tissue-engaging element may be driven longitudinally through the blunt cap.

Turning to FIGS. 1C and 1D it is seen that in some implementations, in an anchored state, tissue-engaging element 156 of tissue anchor 150 extends fully out of blunt cap 130, and into tissue surface 180 at the anatomical site 101, and is screwed into the tissue. In some implementations, head portion 152 of the tissue anchor (e.g., a distal or distal-facing surface 153 thereof) thereby becomes disposed within or against the blunt cap (e.g., against an internal surface 139 of blunt cap 130). That is, cap 130 may become sandwiched (e.g., fixed) against the tissue, and may remain connected to tissue-engaging element 156 and/or head portion 152. Thus, cap 130 may become the head (or part thereof) of anchor 150, disposed (e.g., pressed) against tissue surface 180.

In some implementations, securing blunt cap 130 against the tissue in such a manner can facilitate detachment of the blunt cap from the body of tube 120.

In some implementations, the snap-fit connection between tube 120 and blunt cap 130 can be configured and used to test anchoring of tissue engaging element 156 to the tissue, e.g., by pulling tube 120 proximally.

For example, following driving of tissue-engaging element 156 into tissue surface 180, tube 120 can be pulled proximally away from the tissue. If tissue-engaging element 156 is sufficiently anchored to the tissue, this pulling overcomes the predefined strength of the snap-fit and such that tube 120 becomes detached from blunt cap 130.

By contrast, if tissue-engaging element 156 is not sufficiently anchored, or if the tissue anchor is not fully installed, upon retraction of tube 120, blunt cap 130 will remain attached to the tube and will pull tissue anchor 150 (e.g., tissue-engaging element 156 thereof) along with it. Thus, motion of tube 120 in a proximal direction can be used to verify that tissue anchor 150 has been properly anchored to the tissue.

If it is determined that the anchor has not been properly anchored, it may be retracted proximally through blunt cap 130 and then either withdrawn from the subject, or another attempt to anchor the anchor may be performed.

In some implementations, blunt cap 130 and/or tube 120 may be radiopaque and/or echogenic. In this manner, the detachment can be verified using an imaging technique such as fluoroscopy or echocardiography.

Following anchoring of tissue anchor 150 to the anatomical site 101, catheter 100, tube 120, and driver 140, can be retracted from the anatomical site.

It is to be appreciated that, though FIGS. 1A to 1D show a helical tissue-engaging element of tissue anchor 150, the system and method according to FIGS. 1A to 1D are equally applicable to a tissue anchor having a linear tissue-engaging element, such as a dart, a dowel, a lance, a tine, a barb, etc. Other tissue-engaging elements are also usable, such as one or more of staples, hooks, barbed portions, extensions, etc.

Reference is now made to FIG. 2, which is a schematic sectional illustration of an example system for delivery and installation of a tissue anchor via a delivery tool 202, and of phases of use thereof, in accordance with some implementations. The system of FIG. 2, and the method according to use thereof, can be substantially similar to the system and method according to FIGS. 1A to 1D, with main distinctions described herein.

Delivery tool 202 comprises a longitudinal catheter 200 that is adapted to be advanced toward an anatomical site 201. In some implementations, the anatomical site 201 can be within the heart of the subject (living and/or simulated) or another anatomical site. For example, a distal part 206 of the catheter can be advanced to a chamber (e.g., a left atrium) of the heart.

In some implementations, longitudinal catheter 200 also has an extracorporeal proximal part 207 functionally associated with (e.g., coupled to) a control handle 208 of delivery tool 202 (as illustrated by phase I of FIG. 2). Distal part 206 of the catheter 200 can be guidable to the anatomical site, such as by being actively steerable itself (e.g., by being operatively coupled by one or more pullwires to proximal part 207, such as to a steering controller of control handle 208), or by being passively guided and/or steered (e.g., by extending over or through another steerable element, such as an actively steerable catheter). In some implementations, a longitudinal axis 209 extends between proximal part 207 and steerable distal part 206 of catheter 200.

Catheter 200 can be adapted to be transluminally advanced to the anatomical site 201 using any method known in the art, for example via a vena cava and, if necessary, via the septum separating the atria of the heart and/or via any other blood vessel of the body.

In some implementations, is reversibly attached to distal part 206 of catheter 200, for example by snap fit engagement. The blunt cap 230, includes a proximal wall 232, and a planar end 234. In some implementations, proximal wall 232 of blunt cap 230 is reversibly attached to distal end 206 of catheter 200, for example by snap fit engagement. In some implementations, blunt cap 230 is, or includes a portion, formed of an elastomeric material.

In some implementations, as explained in further detail hereinbelow, the snap fit engagement between catheter 200 and blunt cap 230 has a predefined strength, which can be used to determine whether a tissue anchor has been properly installed.

In some implementations, delivery tool 202 further comprises a driver 240, that terminates in an anchor-engaging interface (e.g., drive head) 242, and extends through catheter 200. In some implementations, anchor-engaging interface 242 is adapted to engage a tissue anchor 250.

In some implementations, driver 240 can be transluminally advanced through catheter 200 and can be controlled by handle 208. As shown, driver 240 can include a longitudinal catheter or shaft configured to extend through a lumen of catheter 200.

In some implementations, tissue anchor 250 includes a head portion 252 defining a driving interface, and a tissue-engaging element 256. In the example shown, tissue-engaging element 256 is a helical tissue-engaging element (e.g., a coil)—e.g., configured to be screwed into the tissue. However, the scope of the present disclosure includes other tissue-engaging elements such as (but not limited to) dart-like, or staple-like tissue-engaging elements.

In some implementations, the driving interface of tissue anchor 250 is adapted to be reversibly engaged by anchor-engaging interface 242 of driver 240. In some implementations, a proximal end of tissue-engaging element 256 is fixedly attached to head portion 252, such that tissue-engaging element 256 is fixed with respect to the driving interface. Tissue-engaging element 256 can be configured to terminate in a sharp distal tip 258.

In some implementations, while the tissue anchor is delivered toward the tissue (e.g., in a delivery state of the system), and prior to driving of the tissue anchor into the tissue, e.g., as shown in phases I and II of FIG. 2, driver 240 and tissue anchor 250 are disposed within catheter 200, and the catheter terminates in distal cap 230, which has an atraumatic surface.

In some implementations, catheter 200 and distal cap 230 are advanced distally toward the anatomical site, as indicated by arrow 270, until planar end 234 of the distal cap engages tissue surface 280 at the anatomical site 201. Additionally, driver 240 and tissue anchor 250 can be advanced distally within catheter 200, such that tip 258 of tissue anchor 250 is adjacent, or engages, the planar end 234 of distal cap 230.

In some implementations, phases III and IV of FIG. 2 illustrate the beginning and end of an anchoring process of the system, respectively. As seen, driver 240 advances tissue anchor 250 distally relative to catheter 100, through blunt cap 230, for example through the center thereof. As such, tissue-engaging element 256 extends distally beyond the blunt cap. In this state, catheter 200 remains attached to blunt cap 230, and head portion 252 of tissue anchor 250 remains within catheter 200 or within blunt cap 230. In some implementations, driver 240 is rotatable, such that rotation of driver 240 causes rotation of tissue anchor 250 (e.g., via head portion 252), thereby screwing tissue-engaging element 256 into tissue surface 280 at anatomical site 201.

In some implementations, at the end of the anchoring process, shown in phase IV, tissue-engaging element 256 of tissue anchor 250 extends fully out of blunt cap 230 and into tissue surface 280. Head portion 252 of the tissue anchor is disposed within the blunt cap, such that a distal surface of the head portion is positioned (e.g., rests) against an internal surface of the blunt cap. In this arrangement, blunt cap 230 is secured, by the head portion 252, against the tissue surface 280, which facilitates detachment of the blunt cap from the body of tube 120.

As shown in phase V, catheter 200 is pulled proximally away from the tissue and the anchored tissue anchor (as indicated by arrow 272), thereby overcoming the predefined strength of the snap-fit engagement between the catheter and blunt cap 230, such that the catheter becomes detached from the blunt cap and the tissue anchor.

If tissue-engaging element 256 is not sufficiently anchored, or if the tissue anchor is not be fully installed, upon retraction of the catheter, blunt cap 230 remains attached to the tube and would pull tissue anchor 250 (e.g., tissue-engaging element 256 thereof) along with it.

In some implementations, blunt cap 230 remains connected to head portion 252, and becomes a part of the anchor head which remains out of tissue surface 280.

In some implementations, the snap-fit connection between catheter 200 and blunt cap 230 is configured and used to test anchoring of the tissue engaging element 256 to the tissue during detachment of the blunt cap from the tube, substantially as described hereinabove with respect to FIGS. 1C and 1D.

Following anchoring of tissue anchor 250 to the anatomical site, catheter 200 and driver 240, can be retracted from the anatomical site.

It should be noted that although FIGS. 1A to 1D and 2 show tissue anchors having a helical tissue-engaging element, the systems and methods illustrated herein are equally applicable to a tissue anchor having a linear tissue-engaging element, such as a dart, a dowel, a lance, a barb, etc. Other tissue engaging elements are also usable, such as one or more hooks, staples, extensions, barbed portions, etc.

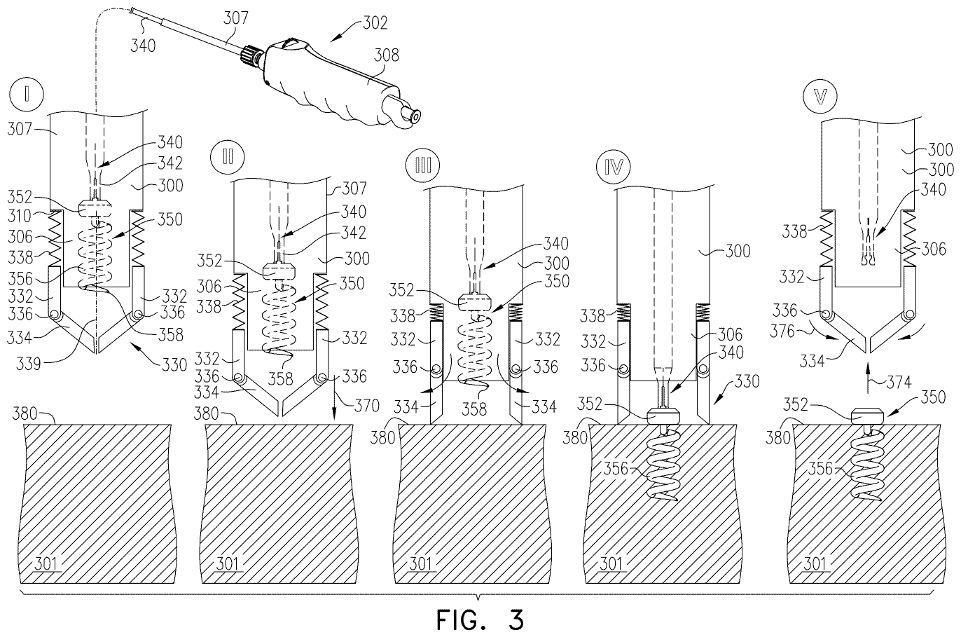

FIGS. 3 to 5 illustrate various implementations in which a tissue anchor can be advanced intracorporeally within a catheter that, at its distal end, has a plurality of segments (e.g., flaps) that form an atraumatic covering (e.g., cap) of the catheter. At the tissue, the segments are pivotable away from each other to form a space therebetween, to allow passage of the tissue anchor therethrough.

Reference is now made to FIG. 3, which is a schematic sectional illustration of an example system comprising a delivery tool 302 for delivery and installation of a tissue anchor, and of phases of use thereof, in accordance with some implementations.

Delivery tool 302 comprises a longitudinal catheter 300 that can be advanced toward an anatomical site 301. In some implementations, the anatomical site can be within the heart of the subject or another location inside a body. For example, a distal part 306 of the catheter can be advanced to a chamber (e.g., a left atrium) of the heart. Catheter 300 also has an extracorporeal proximal part 307 functionally associated with (e.g., coupled to) a control handle 308 of delivery tool 302 (as seen at phase I of FIG. 3).

In some implementations, distal part 306 of the catheter 300 is guidable to the anatomical site, such as by being actively steerable itself (e.g., by being operatively coupled by one or more pullwires to a steering controller at control handle 308), or by being passively guided and/or steered (e.g., by extending over or through another steerable element, such as an actively steerable catheter). A longitudinal axis 309 can extend between proximal part 307 and steerable distal part 306 of catheter 300.

In some implementations, catheter 300 can be adapted to be transluminally advanced to the anatomical site using any method known in the art, for example via a vena cava and, if necessary, via the septum separating the atria of the heart and/or via any other blood vessel of the body.

In some implementations, distal part 306 of longitudinal catheter 300 has a slightly smaller diameter than the remainder of catheter 300, such that a shoulder 310 is formed on an exterior surface of catheter 300, just proximally to distal part 306.

In some implementations, a blunt cap 330 is attached to distal part 306 of catheter 300. The blunt cap 330 can include a proximal wall 332, and a plurality of flaps 334 (e.g., segments) pivotally attached to proximal wall 332 at pivot points 336. In some implementations, flaps 334 are configured such to point in a medial direction and/or converge (e.g., the state of the flaps illustrated in phases I-II of FIG. 3), yet the flaps can pivot to be aligned with, or flush with, proximal wall 332 when a sufficient force is applied (e.g., the state of the flaps illustrated in phases III-IV of FIG. 3).

In some implementations, proximal wall 332 can be a circumferential proximal wall. In some implementations, proximal wall 332 can include a plurality of proximal wall segments, each associated with one of flaps 334.

In some implementations, proximal wall 332 is attached to catheter 300 by a compression spring 338, e.g., a circumferential spring. In some implementations, the compression spring is seated against shoulder 310 and is disposed about an exterior of distal part 306 of the catheter 300.

In a relaxed state of spring 338, the spring can push proximal wall 332 in a distal direction, such that pivot point 336 is disposed distally beyond the distal end of catheter 300. In such a relaxed state of the spring, flaps 334 are in a rest state, in which the flaps point radially inward and form an atraumatic distal cap over the catheter.

In some implementations, proximal wall 332 is a continuation of catheter 300 (e.g., the catheter and the proximal wall are formed from the same tube).

In some implementations, delivery tool 302 further comprises a driver 340 that terminates in a distal driving tool 342, and that extends through catheter 300. In some implementations, driver 340 can be transluminally advanced through catheter 300, and can be controlled by handle 308. Driver 340 may be a variant of, or substantively identical to, any of drivers 140 and/or 240.

In some implementations, driving tool 342 includes an anchor-engaging interface, adapted to engage a tissue anchor 350. As shown, driver 340 can include a longitudinal catheter (e.g., a control rod) extending through a lumen of catheter 300.

Similarly to tissue anchors 150 and/or 250, tissue anchor 350 can include a head portion 352 defining a driving interface, and a tissue-engaging element 356. In the example shown, tissue-engaging element 356 is a helical tissue-engaging element—e.g., configured to be screwed into the tissue. However, the scope of the present disclosure includes other tissue-engaging elements such as (but not limited to) dart-like, or staple-like tissue-engaging elements.

The driving interface of tissue anchor 350 is adapted to be reversibly engaged by the anchor-engaging interface of driver 340. In some implementations, a proximal end of tissue-engaging element 356 is fixedly attached to head portion 352, and such that tissue-engaging element 356 is fixed with respect to the driving interface. Tissue-engaging element 356 can be configured to terminate in a sharp distal tip 358.

In some implementations, during delivery of tissue anchor 350 towards the heart (e.g., shown at phases I-II of FIG. 3), driver 340 and tissue anchor 350 are disposed within catheter 300, (e.g., within distal part 306). As described hereinabove, in this state, flaps 334 are in a rest state, in which the flaps point in a medial direction and/or converge, thereby forming an atraumatic distal cap 330 for the catheter. Catheter 300 and distal cap 330 are advanced distally toward the anatomical site, in the direction of arrow 370, until tips of flaps 334 of the distal cap engages a tissue surface 380 at the anatomical site.

Turning to phase III, in some implementations, once cap 330 (e.g., flaps 334 thereof) abut anatomical site 301, and are thereby prevented from moving further distally, spring 338 strains (e.g., compresses) to allow catheter 300 to continue to move distally. This further distal movement of catheter 300 moves distal part 306 of the catheter between flaps 334, pushing them to pivot in a lateral direction, as indicated by arrows 372-e.g., until the flaps are aligned with, or flush with, proximal wall 332. At this point, the tissue anchor can be anchored into the tissue (phase IV).

Phase IV of FIG. 3 illustrates tissue anchor 350 anchored in the tissue, e.g., at the end of the anchoring process of the system. As seen, driver 340 has advanced tissue anchor 350 distally through the now open flaps 334. In some implementations, driver 340 is rotatable, such that rotation of driver 340 screws tissue-engaging element 356 into tissue surface 380 at the anatomical site (e.g., via rotation of head portion 352), such that at the end of the anchoring process, tissue-engaging element 356 of tissue anchor 350 extends fully into anatomical site 301. Head portion 352 of the tissue anchor can be disposed outside the tissue, e.g., such that a distal surface of the head portion rests against surface 380.

Following anchoring of tissue anchor 350 to the anatomical site 301, catheter 300 can be retracted in a proximal direction from tissue surface 380, as illustrated in phase V, and indicated by arrow 374. That is, the catheter 300 can be retracted away from the anatomical site and out of the subject. Retracting the catheter 300 allows spring 338 to decompress and move distal part 306 of the catheter proximally relative to flaps 334. As a result, pressure is no longer applied by distal part 306 to flaps 334, and the flaps pivot about pivot points 336, in a medial direction indicated by arrows 376, to re-form the atraumatic cap over the catheter.

Driver 340 can similarly be retracted away from the anatomical site (e.g., along with catheter 300) and out of the subject.

It is to be understood that although FIG. 3 show a helical tissue-engaging element of tissue anchor 350, the system and method according to FIG. 3 are equally applicable to a tissue anchor having a linear tissue-engaging element, such as a dart, a dowel, a lance, etc. Other tissue-engaging elements are also usable, such as one or more hooks, staples, barbed-portions, extensions, arms, etc.

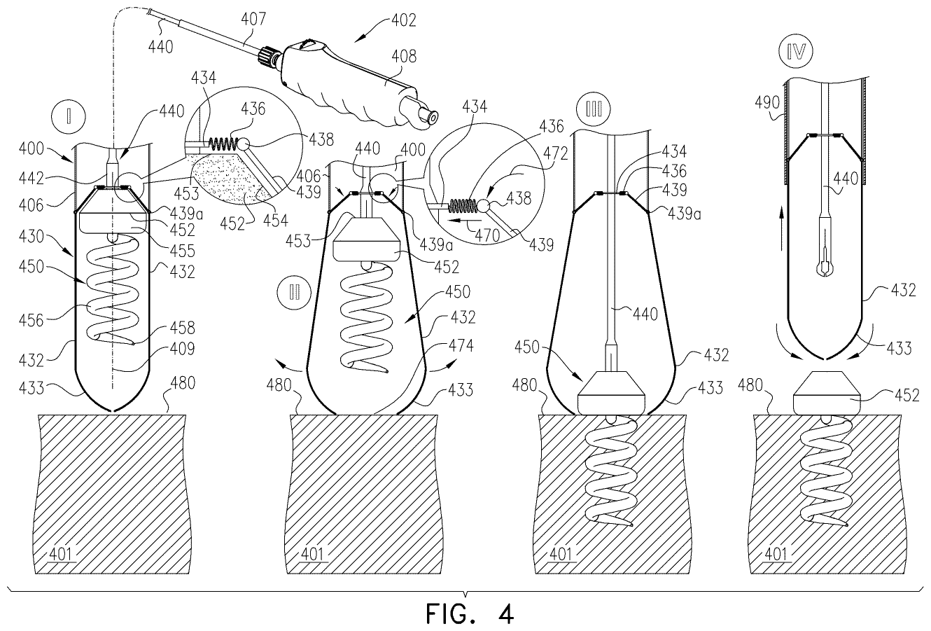

Reference is now made to FIG. 4, which is a schematic sectional illustration of an example system comprising a delivery tool 402 for delivery and installation of a tissue anchor, and of phases of use thereof, in accordance with some implementations.

In some implementations, as seen at phase I of FIG. 4, delivery tool 402 comprises a longitudinal catheter 400 that is adapted to be advanced toward an anatomical site 401. In some implementations, the anatomical site can be within the heart of the subject. For example, a distal part 406 of the catheter can be advanced to a chamber (e.g., a left atrium) of the heart. In some implementations, longitudinal catheter 400 also has an extracorporeal proximal part 407 functionally associated with (e.g., coupled to) a control handle 408 of delivery tool 402.

In some implementations, distal part 406 of catheter 400 is guidable to the anatomical site, such as by being actively steerable itself (e.g., by being operatively coupled by one or more pullwires to a steering controller that is at control handle 408), or by being passively guided and/or steered (e.g., by extending over or through another steerable element, such as an actively steerable catheter). A longitudinal axis 409 extends between proximal part 407 and steerable distal part 406 of catheter 400.

Catheter 400 can be adapted to be transluminally advanced to the anatomical site using any method known in the art, for example via the vena cava and, if necessary, via the septum separating the atria of the heart.

In some implementations, distal part 406 of catheter 400 is attached to, or comprises, a blunt cap 430. In some implementations, blunt cap 430 comprises and/or is formed from a plurality of segments, or flaps, 432, attached to a spring loading mechanism. In some implementations, each flap 432 terminates at a distal end 433, which points radially inwardly.

In some implementations, the spring loading mechanism includes a central ring 434, having a plurality of extension springs 436 disposed in a radially outward direction therefrom. In some implementations, each extension spring 436 terminates, in a radially outward end thereof, in an end point 438.

In some implementations, an arm 439, which is pivotable about a pivot point 439a, connects each such end point to a corresponding flap 432.

In some implementations, each flap 432 and corresponding arm 439 together form a structure that is pivotally coupled to the distal end of the catheter 400 and is biased to pivot, about pivot point 439a, such that arm 439 deflects medially and flap 432 deflect laterally.

In some implementations, delivery tool further comprises a driver 440 that terminates in a distal driving tool 442. The distal driving tool 442 can extend through catheter 400 and through central ring 434 of cap 430. Driver 440 may be a variant of, or substantively identical to, any of the drivers disclosed herein.

In some implementations, driver 440 can be transluminally advanced through catheter 400 and can be controlled by handle 408. In some implementations, driving tool 442 includes an anchor-engaging interface, adapted to engage a tissue anchor 450. As shown, driver 440 can include a longitudinal catheter (e.g., a control rod) extending through a lumen of catheter 400.

In some implementations, tissue anchor 450 includes a head portion 452 defining a driving interface, and a tissue-engaging element 456. In the example shown, tissue-engaging element 456 is a helical tissue-engaging element—e.g., configured to be screwed into the tissue. However, the scope of the present disclosure includes other tissue-engaging elements such as (but not limited to) dart-like, or staple-like tissue-engaging elements. In some implementations, head portion 452 can include a planar proximal surface 453, from which extends a sloped surface 454 (e.g., a bevel or chamfer) to a base portion 455.

In some implementations, the driving interface of tissue anchor 450 is adapted to reversibly engage the anchor-engaging interface of driver 440.

In some implementations, proximal end of tissue-engaging element 456 is fixedly attached to head portion 452, and such that tissue-engaging element 456 is fixed with respect to the driving interface. Tissue-engaging element 456 can be configured to terminate in a sharp distal tip 458.

In some implementations, during delivery of tissue anchor 450 towards the heart (e.g., shown at phase I of FIG. 4), driver 440 is disposed within catheter 400 through central ring 434, and is connected to tissue anchor 450 which is disposed within blunt cap 430. In some implementations, head portion 452 of tissue anchor 450 is disposed adjacent ring 434.

In some implementations, proximal surface 453 of head portion 452 is wider than ring 434 and extension springs 436, when the springs are in their resting state. Accordingly, the proximal end of head portion 452 can apply a force to extension springs 436, constraining them in a strained (e.g., extended) state. In some implementations, in this arrangement, head portion 452 obstructs arms 439 from deflecting medially and the flaps 432 from pivoting, thereby holding flaps 432 medially and substantially flush with catheter 400, with distal ends 433 substantially engaging one another and forming an atraumatic tip, or cap, of the catheter.

Phase I shows catheter 400 having been advanced toward anatomical site 401, such that distal ends 433 of blunt cap 430 are close to tissue surface 480. Driver 440 can then begin advancing tissue anchor 450 in a distal direction, toward tissue surface 480 (Phase II).

As illustrated by the transition between Phase I and Phase II of FIG. 4, advancing driver 440, and therefore anchor 450, in a distal direction causes head portion 452 to cease applying force on springs 436, thereby allowing the springs to contract in a medial direction toward their resting state, as indicated by arrow 470. Contraction of the springs can cause arms 439 to pivot about corresponding pivot points 439a in a medial direction, indicated by arrow 472.

In some implementations, flaps 432 are fixedly attached to arms 439 (or the arms can simply be extensions of the flaps), such that the pivoting of arms 439 causes pivoting of flaps 432 in a radially outward direction. The pivoting of flaps 432 radially outward may form space 474 between distal ends 433 of the flaps (e.g., for passage of tissue anchor 450 therethrough).

At this point, the tissue anchor can be advanced through space 474 (e.g., between the now open flaps 432) and into tissue 401 (e.g., as shown in phase III of FIG. 4). As described hereinabove with reference to drivers 140, 240, and/or 340, in some implementations, driver 440 is rotatable, such that rotation of driver 440 causes rotation of head portion 452, and screws tissue-engaging element 456 into tissue surface 480 at the anatomical site.

At the end of the anchoring process tissue-engaging element 456 of tissue anchor 450 can extend fully into anatomical site 401. In some implementations, head portion 452 of the tissue anchor is disposed outside the tissue, such that a distal surface of the head portion rests against surface 480.

Phase IV represents the anchored state of the system, with catheter 400 having been retracted in a proximal direction from tissue surface 480, as indicated by arrow 476. Phase IV illustrates catheter 400 retracted into a cannula (e.g., sheath) 490, such that flaps 432 pivot back into their original position. For example, cannula 490 may press medially against an external surface of flaps 432, pushing the flaps medially.

Following anchoring of tissue anchor 450 to the anatomical site, catheter 400 and driver 440, can be retracted from the anatomical site.

It is to be noted that although FIG. 4 show a helical tissue-engaging element of tissue anchor 450, the system and method according to FIG. 4 are equally applicable to a tissue anchor having a linear tissue-engaging element, such as a dart, a dowel, a lance, etc. Other tissue engaging elements are also usable, such as one or more hooks, staples, extensions, arms, barbed portions, etc.

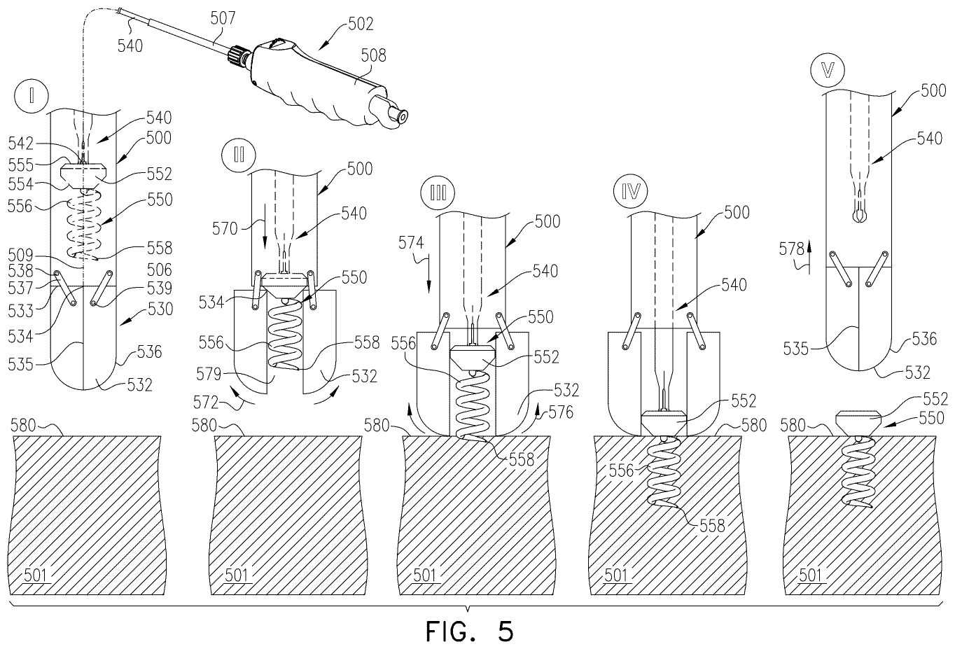

Reference is now made to FIG. 5, which is a schematic sectional illustration of an example system comprising a delivery tool 502 for delivery and installation of a tissue anchor, and of phases of use thereof, in accordance with some implementations.

As seen at phase I of FIG. 5, delivery tool 502 comprises a longitudinal catheter 500 that is advanced toward an anatomical site 501. In some implementations, the anatomical site 501 can be within the heart of the subject. For example, a distal part 506 of the catheter can be advanced to a chamber (e.g., a left atrium) of the heart. Longitudinal catheter 500 also has an extracorporeal proximal part 507 functionally associated with a control handle 508 of delivery tool 502.

In some implementations, distal part 506 of catheter 500 is guidable to the anatomical site 501, such as by being actively steerable itself (e.g., by being operatively coupled by one or more pullwires to a steering controller at control handle 508), or by being passively guided and/or steered (e.g., by extending over or through another steerable element, such as an actively steerable catheter). A longitudinal axis 509 extends between proximal part 507 and steerable distal part 506 of catheter 500.

In some implementations, catheter 500 can be adapted to be transluminally advanced to the anatomical site using any method known in the art, for example via the vena cava and, if necessary, via the septum separating the atria of the heart and/or via any other blood vessels of the subject.

In some implementations, a blunt cap 530 is attached to (e.g., may be formed by) distal part 506 of catheter 500. In some implementations, blunt cap 530 includes a plurality of curved segments 532, here shown as a pair of segments. In some implementations, each segment 532 includes a substantially horizontal upper surface 533, attached at a corner 534 to a substantially vertical central surface 535.

In some implementations, a curved surface 536 extends from a radially outward end of upper surface 533 to a distal end of central surface 535. One of the curved segments 532 can be pivotally attached to distal part 506 of catheter 500 by pivoting bar 537 (e.g., each curved segment can be attached by at least one pivoting bar). In some implementations, each pivoting bar 537 is attached at a first pivot point 538 to the distal part 506 of the catheter, and at a second pivoting point 539 to a proximal part of one of segments 532.

In some implementations, segments 532 and pivoting bars 537 are configured such that the segments are biased to engage each other along the longitudinal axis 509 and the curve of segments 532 forms a curved atraumatic tip of the system (e.g., catheter). In some implementations, segments 532 can pivot radially outward and/or longitudinally upward, when a suitable force is applied. Such pivoting exposes, or opens, distal part 506 of catheter 500.

In some implementations, delivery tool 502 further comprises a driver 540 that terminates in a distal driving tool 542, and that extends through catheter 500. In some implementations, driver 540 can be transluminally advanced through catheter 500, and can be controlled by handle 508. In some implementations, driving tool 542 includes an anchor- engaging interface, adapted to engage a tissue anchor 550. As shown, driver 540 can include a longitudinal catheter extending through a lumen of catheter 500.

In some implementations, tissue anchor 550 includes a head portion 552 defining a driving interface, and a tissue-engaging element 556. In the example shown, tissue-engaging element 556 is a helical tissue-engaging element—e.g., configured to be screwed into the tissue. However, the scope of the present disclosure includes other tissue-engaging elements such as (but not limited to) dart-like, or staple-like tissue-engaging elements.

In some implementations, head portion 552 includes a distal base 553, extending proximally to a sloped portion 554, and terminating in a proximal base 555. In some implementations, proximal base 555 has a greater diameter than distal base 553.

In some implementations, the driving interface of tissue anchor 550 is adapted to reversibly engage the anchor-engaging interface of driver 540. In some implementations, a proximal end of tissue-engaging element 556 is fixedly attached to head portion 552, and such that tissue-engaging element 556 is fixed with respect to the driving interface. In some implementations, tissue-engaging element 556 can be configured to terminate in a sharp distal tip 558.

In some implementations, during transluminal delivery of tissue anchor 550 towards the heart (e.g., shown at phase I of FIG. 5), driver 540 and tissue anchor 550 are disposed within catheter 500. In some implementations, pivoting bars 537 and segments 532 are in their resting state, such that upper surfaces 533 of segments 532 engage the distal end of catheter 500, and central surfaces 535 of segments 532 engage one another along longitudinal axis 509. As such, external surfaces 536 segments 532 form an atraumatic distal cap over the catheter. In this arrangement, tissue anchor 550 can be disposed within catheter 500, as shown, or can be disposed substantially within blunt cap 530.

In some implementations, at phase II, driver 540 begins driving tissue anchor 550 in a distal direction relative to catheter 500, as indicated by arrow 570. In some implementations, tissue-engaging element 556 enters blunt cap 530 until distal base 553 of head portion 552 pushes against an interior of upper surfaces 533, forcing segments 532 downwards, and away from each other, to form an opening 579 between the segments.

In some implementations, as shown in phase III, driver 540 continues driving of tissue anchor 550 in a distal direction relative to catheter 500, as indicated by arrow 574. This causes corners 534 of segments 532 to engage, and slide against, sloped portion 554 of head portion 552 of tissue anchor 550. As a result, segments 532 pivot further outwardly, in the direction of arrows 576.

In some implementations, the enlarged opening 579 can have a width substantially equal to the greatest width of head portion 552, such that the head portion can slide along the central surfaces 535 of segments 532. In some implementations, in phase III, blunt cap 530 and tissue anchor 550 have also been advanced such that ends of blunt cap 530 engage tissue surface 580, to initiate anchoring of tissue anchor 550.

In some implementations, driver 540 is rotatable, such that rotation of driver 540 causes rotation of head portion 552 and causes distal tip 558 of tissue anchor 550 to be screwed into tissue surface 580.

In some implementations, at phase IV, driver 540 has advanced tissue anchor 550 distally into anatomical site 501, until the distal base 553 of head portion 552 engages tissue surface 580.

In some implementations, in phase V, which represents the anchored state of the system, catheter 500 has been retracted in a proximal direction from tissue surface 580, as indicated by arrow 578. This removes the force from segments 532, allowing them to pivot back to their resting state, in which central surfaces 535 engage one another.

Following anchoring of tissue anchor 550 to the anatomical site, catheter 500 and driver 540, can be retracted from the anatomical site.

In some implementations, though FIG. 5 show a helical tissue-engaging element of tissue anchor 550, the system and method according to FIG. 5 are equally applicable to a tissue anchor having a linear tissue-engaging element, such as a dart, a dowel, a lance, etc. Other tissue-engaging elements are also usable, such as one or more hooks, staples, arms, extensions, barbed-portions, etc.

In the description of FIGS. 2 to 5 hereinabove, the blunt caps are described as being attached to the distal end of the catheter. However, it is to be appreciated that the blunt caps of FIGS. 2 to 5 can be attached to a tube, or capsule, similar to tube 120 of FIG. 1. The systems of FIGS. 2 to 5 would function in a similar manner if such a tube were used. Similarly, although the system of FIG. 1 illustrates a blunt cap at the end of a tube that is disposed at a distal end of a catheter, it is to be understood that the blunt cap of FIG. 1 may simply be used to cover a distal end of a catheter directly (e.g., without a tube therebetween).

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, apparatus, etc. (e.g., with heat, radiation, ethylene oxide, hydrogen peroxide, etc.).

For the purposes of this specification and the claims that follow, the term “substantially” is defined as “at least 95%” of the related quantity. For example, “substantially perpendicular” means at least 95% perpendicular, or having an angle in the range of 85° to 95°.

It should be understood that the use of “and/or” is defined inclusively such that the term “a and/or b” should be read to include the sets: “a and b,” “a or b,” “a,” “b.”

The present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the treatment techniques, methods, operations, steps, etc. described or suggested herein can be performed on a living subject (e.g., human, other animal, etc.) or on a non-living subject (e.g., a simulation, such as a cadaver, cadaver heart, simulator, anthropomorphic phantom, etc.). When performed on a simulation, the body parts, e.g., heart, tissue, valve, etc., can optionally be referred to as “simulated” (e.g., simulated heart, simulated tissue, simulated valve, etc.) and can comprise computerized and/or physical representations of the body parts, tissue, etc.)

### Example Implementations (Some Non-Limiting Examples of the Concepts Herein Are Recited Below)

Example 1. A system usable and/or for use with a tissue at a real or simulated anatomical site of a subject, the system comprising: (A) a tissue anchor, including a sharp tissue-engaging element and a driving interface which is fixed with respect to the tissue-engaging element; and/or (B) a delivery tool comprising: (i) a tube, having a blunt cap reversibly attached to and covering a distal end of the tube, the tube being transluminally advanceable in a distal direction toward the anatomical site; and/or (ii) a driver that, while engaged with the driving interface of the tissue anchor, is configured to drive the tissue-engaging element through the blunt cap and into the tissue, such that the blunt cap becomes an anchor-head of the tissue anchor, secured to the tissue by the tissue-engaging element.

Example 2. The system according to example 1, wherein: (i) the tube defines a protrusion, and the blunt cap defines an indentation, and/or (ii) the blunt cap is reversibly attached to the tube by the protrusion being disposed within the indentation.

Example 3. The system according to any one of examples 1 to 2, wherein: (i) the blunt cap defines a protrusion, and the tube defines an indentation, and/or (ii) the blunt cap is reversibly attached to the tube by the protrusion being disposed within the indentation.

Example 4. The system according to any one of examples 1 to 3, wherein the driver is transluminally advanceable, within the tube, in the distal direction towards the anatomical site.

Example 5. The system according to any one of examples 1 to 4, wherein the tube is transluminally advanceable, in the distal direction towards the anatomical site, while the tissue anchor is disposed within the tube, proximally from the blunt cap.

Example 6. The system according to any one of examples 1 to 5, wherein the delivery tool further comprises a longitudinal catheter, and wherein the tube is disposed at a distal end of the longitudinal catheter.

Example 7. The system according to example 6, wherein the tube is attached to a distal end of the longitudinal catheter.

Example 8. The system according to any one of examples 1 to 7, wherein the blunt cap includes a predefined opening for passage of the tissue-engaging element of the tissue anchor through the blunt cap.

Example 9. The system according to any one of examples 1 to 8, wherein the blunt cap is pierceable by the tissue-engaging element during driving of the tissue-engaging element through the blunt cap of the tube.

Example 10. The system according to any one of examples 1 to 9, wherein: (i) the tissue-engaging element comprises a helical tissue-engaging element, and/or (ii) the driver is configured to helically drive the helical tissue-engaging element through the blunt cap and into the tissue via application of torque to the driver.

Example 11. The system according to any one of examples 1 to 10, wherein the tissue-engaging element comprises a linear tissue-engaging element, adapted to be driven longitudinally through the blunt cap.

Example 12. The system according to example 11, wherein the linear tissue- engaging element comprises a dart or a dowel.

Example 13. The system according to any one of examples 1 to 12, wherein the blunt cap is attached to the tube by snap fit engagement, having a predefined strength.

Example 14. The system according to example 13, wherein the snap fit engagement is configured such that driving the tissue-engaging element through the blunt cap and into the tissue facilitates detachment of the blunt cap from the tube.

Example 15. The system according to example 13, wherein the delivery tool is adapted to test anchoring of the tissue-engaging element to the tissue following driving of the tissue-engaging element through the blunt cap, via pulling of the tube such that (i) sufficient anchoring of the tissue-engaging element to the tissue results in the pulling of the tube overcoming the predefined strength and thereby detaching the tube from the blunt cap, whereas (ii) insufficient anchoring of the tissue-engaging element to the tissue results in the pulling of the tube pulling the blunt cap and the tissue anchor away from the tissue.

Example 16. The system according to example 15, wherein the tissue-engaging element and the blunt cap are configured such that the driver can retract the tissue-engaging element proximally through the blunt cap.

Example 17. A system usable and/or for use with a subject, the system comprising: (A) a tissue anchor, including a sharp tissue-engaging element and a driving interface which is fixed with respect to the tissue-engaging element; and/or (B) a delivery tool comprising: (i) a tube, having a blunt cap covering a distal end of the tube, and configured to be transluminally advanced toward an anatomical site of the subject with the tissue anchor disposed within the tube, proximally from the blunt cap; and/or (ii) a driver, extending through the tube while engaged with the driving interface of the tissue anchor, and configured to advance the tissue-engaging element distally beyond the cap, and to anchor the tissue anchor at the anatomical site by driving the tissue-engaging element into tissue at the anatomical site.

Example 18. The system according to example 17, wherein the delivery tool further comprises a longitudinal catheter, and wherein the tube is at a distal end of the longitudinal catheter.

Example 19. The system according to example 18, wherein the tube comprises a sheath attached to a distal end of the longitudinal catheter.

Example 20. The system according to any one of examples 17 to 19, wherein the blunt cap is integrally formed with the tube.

Example 21. The system according to any one of examples 17 to 20, wherein the blunt cap is attached to the distal end of the tube.

Example 22. The system according to example 21, wherein the tissue-engaging element of the tissue anchor is adapted to be driven through the blunt cap of the tube and into the tissue, such that the anchoring of the tissue anchor to the tissue secures the blunt cap against the tissue, thereby facilitating detachment of the blunt cap from the tube, and converting the blunt cap into an anchor-head of the tissue anchor.

Example 23. The system according to example 22, wherein the blunt cap includes a predefined opening for passage of the tissue-engaging element of the tissue anchor through the blunt cap.

Example 24. The system according to example 22, wherein the blunt cap is pierceable by the tissue-engaging element during driving of the tissue-engaging element through the blunt cap of the tube.

Example 25. The system according to example 22, wherein the tissue-engaging element comprises a helical tissue-engaging element, adapted to be helically driven through the blunt cap.

Example 26. The system according to example 22, wherein the tissue-engaging element comprises a linear tissue-engaging element, adapted to be driven longitudinally through the blunt cap.

Example 27. The system according to example 26, wherein the linear tissue- engaging element comprises a dart or a dowel.

Example 28. The system according to example 22, wherein the blunt cap is attached to the tube by a snap fit engagement that has a predefined strength.

Example 29. The system according to example 28, wherein the delivery tool is adapted to test anchoring of the tissue-engaging element to the tissue following driving of the tissue-engaging element through the blunt cap, via pulling of the tube such that (i) sufficient anchoring of the tissue-engaging element to the tissue results in the pulling of the tube overcoming the predefined strength and thereby detaching the tube from the blunt cap, whereas (ii) insufficient anchoring of the tissue-engaging element to the tissue results in the pulling of the tube pulling the cap and the tissue anchor away from the tissue.

Example 30. The system according to example 29, wherein the tissue-engaging element and the cap are configured such that the driver can retract the tissue-engaging element proximally through the blunt cap.

Example 31. The system according to any one of examples 17 to 30, wherein the blunt cap is defined by a plurality of segments that are adapted to pivot laterally away from each other in a manner that facilitates the driver advancing the tissue-engaging element distally beyond the cap.

Example 32. The system according to example 31, wherein the segments are mounted on the tube such that, upon advancement of the delivery tool distally in a manner that presses the cap against the tissue, the segments responsively pivot laterally away from each other in the manner that facilitates the driver advancing the tissue-engaging element distally beyond the cap.

Example 33. The system according to example 32, wherein the segments are mounted on the tube such that, upon advancement of the delivery tool distally in a manner that presses the cap against the tissue, a distal portion of the catheter advances distally between the segments, pushing the segments to pivot laterally away from each other in the manner that facilitates the driver advancing the tissue-engaging element distally beyond the cap.

Example 34. The system according to example 33, wherein the segments are biased to pivot medially toward each other.

Example 35. The system according to example 31, wherein: (i) each of the segments is pivotably attached to the distal end of the tube; and/or (ii) the delivery tool is configured such that advancement of the driver distally with respect to the segments pushes the segments to pivot laterally away from each other in a manner that forms an opening through which the driver is configured to advance the tissue anchor distally beyond the cap.

Example 36. The system according to example 35, wherein the tissue anchor includes a head portion having a sloped surface that is dimensioned such that the advancement of the driver distally with respect to the segments slides the sloped surface along the segments in a manner that pushes the segments to pivot laterally away from each other.

Example 37. The system according to example 31, wherein: (i) for each of the segments, a respective arm is fixed to the segment to form a structure that is pivotably coupled to the distal end of the tube such that (i) pivoting of the structure in a first direction deflects the arm medially and deflects the segment laterally, and (ii) pivoting of the structure in a second direction deflects the arm laterally and deflects the segment medially; (ii) in a delivery state of the system, a head portion of the tissue anchor is disposed against each arm in a manner that obstructs the arms from deflecting medially, thereby preventing each structure from pivoting in the first direction; and/or (iii) each of the structures is biased to pivot in the first direction such that, upon advancement of the tissue anchor distally away from the arms, each of the structures responsively pivots in the first direction, such that the segments deflect laterally, thereby forming an opening through which the driver is configured to advance the tissue anchor distally beyond the cap.

Example 38. A system usable and/or for use with a subject, the system comprising: (A) a tissue anchor, including a sharp tissue-engaging element and a driving interface which is fixed with respect to the tissue-engaging element; (B) a tube, having a blunt cap reversibly attached to and covering a distal end of the tube, the tube configured to be transluminally advanced toward an anatomical site of the subject with the tissue anchor disposed within the tube; and/or (C) a driver, extending through the tube, and configured to anchor the tissue anchor at the anatomical site by driving the tissue-engaging element into tissue at the anatomical site, wherein the tissue-engaging element of the tissue anchor is adapted to be driven through the blunt cap of the tube and into the tissue, such that the anchoring of the tissue anchor to the tissue secures the blunt cap against the tissue, thereby facilitating detachment of the blunt cap from the tube, and converting the blunt cap into an anchor-head of the tissue anchor

Example 39. A method for anchoring a tissue anchor having a tissue-engaging element in real or simulated tissue at a real or simulated anatomical site of a real or simulated subject, the method comprising: (A) transluminally advancing a tube, having a blunt cap closing a distal end of the tube, toward the anatomical site of the subject; (B) advancing the tissue-engaging element distally beyond the blunt cap; and/or (C) anchoring the tissue anchor at the anatomical site by driving the tissue-engaging element into tissue at the anatomical site.

Example 40. The method according to example 39, wherein the advancing of the tissue-engaging element distally beyond the blunt cap comprises driving the tissue-engaging element through the blunt cap of the tube and into the tissue, the method further comprising, following the anchoring of the tissue anchor, converting the blunt cap into an anchor-head of the tissue anchor by detaching the blunt cap from the tube.

Example 41. The method according to example 40, wherein the blunt cap includes a predefined opening for passage of the tissue-engaging element of the tissue anchor, and wherein the driving of the tissue-engaging element through the blunt cap comprises driving the tissue-engaging element through the predefined opening.

Example 42. The method according to example 40, wherein the driving of the tissue-engaging element through the blunt cap comprises, using the tissue-engaging element, piercing a hole through the blunt cap, for the tissue-engaging element to extend distally beyond the blunt cap.

Example 43. The method according to example 40, wherein the tissue-engaging element comprises a helical tissue-engaging element, and the driving of the tissue-engaging element comprises helically driving the tissue-engaging element through the blunt cap.

Example 44. The method according to example 40, wherein the tissue-engaging element comprises a linear tissue-engaging element, and the driving of the tissue-engaging element comprises longitudinally driving the tissue-engaging element through the blunt cap.

Example 45. The method according to example 40, further comprising, following driving of the tissue-engaging element into the tissue, pulling the tube proximally to test anchoring of the tissue-engaging element to the tissue, such that (i) sufficient anchoring of the tissue-engaging element to the tissue results in the pulling of the tube overcoming the predefined strength and thereby detaching the tube from the blunt cap, whereas (ii) insufficient anchoring of the tissue-engaging element to the tissue results in the pulling of the tube pulling the cap and the tissue anchor away from the tissue.

Example 46. The method according to example 45, further comprising retracting the tissue-engaging element proximally through the blunt cap if pulling of the tube does not overcome the predefined strength to detach the blunt cap from the tube.

Example 47. The method according to any one of examples 39 to 46, wherein the blunt cap comprises a plurality of segments forming a substantially tapered structure, and wherein the advancing of the tissue-engaging element distally beyond the blunt cap comprises moving the segments outwardly in a radial direction to expose the tissue-engaging element of the tissue anchor for anchoring thereof.

Example 48. The method according to example 47, wherein the moving of the segments comprises causing the segments to pivot outwardly in the radial direction.

Example 49. A method for anchoring a tissue anchor having a tissue-engaging element in a real or simulated tissue at a real or simulated anatomical site of a real or simulated subject, the method comprising: (A) transluminally advancing a tube, having a blunt cap reversibly attached to and closing a distal end of the tube, toward the anatomical site of the subject; (B) anchoring the tissue anchor at the anatomical site by driving the tissue-engaging element through the blunt cap of the tube and into tissue at the anatomical site; and/or (C) converting the blunt cap into an anchor-head of the tissue anchor by detaching the tube from the blunt cap.

Example 50. The method according to example 49, wherein the blunt cap includes a predefined opening for passage of the tissue-engaging element of the tissue anchor through the blunt cap, and wherein the driving of the tissue-engaging element through the blunt cap comprises driving the tissue-engaging element through the predefined opening.

Example 51. The method according to any one of examples 49 to 50, wherein the driving of the tissue-engaging element through the blunt cap comprises, using the tissue-engaging element, piercing a hole through the blunt cap, for the tissue-engaging element to extend distally beyond the blunt cap.

Example 52. The method according to any one of examples 49 to 51, wherein the tissue-engaging element comprises a helical tissue-engaging element, and the driving of the tissue-engaging element comprises helically driving the tissue-engaging element through the blunt cap.

Example 53. The method according to any one of examples 49 to 52, wherein the tissue-engaging element comprises a linear tissue-engaging element, and the driving of the tissue-engaging element comprises longitudinally driving the tissue-engaging element through the blunt cap.

Example 54. The method according to any one of examples 49 to 53, wherein the detaching comprises testing anchoring of the tissue-engaging element to the tissue by drawing the tube proximally away from the tissue, such that only if the tissue-engaging element is sufficiently anchored to the tissue, the drawing of the tube proximally away from the tissue overcomes a predefined strength to detach the blunt cap detaches from the tube.

Example 55. The method according to example 54, further comprising retracting the tissue-engaging element proximally through the blunt cap if the drawing of the tube does not overcome the predefined strength to detach the blunt cap from the tube.

Example 56. The method according to example 39, wherein: (i) the blunt cap is defined by a plurality of segments that are adapted to pivot laterally away from each other, (ii) advancing the tube toward the anatomical site comprises advancing the tube toward the anatomical site while the segments are pivoted medially toward each other, and/or (iii) advancing the tissue-engaging element distally beyond the blunt cap comprises advancing the tissue-engaging element distally beyond the blunt cap while the segments are pivoted laterally away from each other.

Example 57. The method according to example 56, wherein: (i) the segments are mounted on the tube such that, upon advancement of the tube distally in a manner that presses the cap against the tissue, the segments responsively pivot laterally away from each other in the manner that facilitates the advancing of the tissue-engaging element distally beyond the blunt cap, and/or (ii) advancing the tube toward the anatomical site comprises advancing the tube toward the anatomical site such that the cap presses against the tissue.

Example 58. The method according to example 57, wherein: (i) the segments are mounted on the tube such that, upon advancement of the tube distally in a manner that presses the cap against the tissue, a distal portion of the catheter advances distally between the segments, pushing the segments to pivot laterally away from each other, and/or (ii) advancing the tissue-engaging element distally beyond the blunt cap comprises advancing the tissue-engaging element distally beyond the blunt cap while the segments are pushed laterally away from each other.

Example 59. The method according to example 58, wherein: (i) the segments are biased to pivot medially toward each other, and/or (ii) advancing the tissue-engaging element distally beyond the blunt cap comprises advancing the tissue-engaging element distally beyond the blunt cap while the segments are pivoted laterally away from each other.

Example 60. The method according to example 56, wherein: (i) each of the segments is pivotably attached to the distal end of the tube; and/or (ii) the method further comprises, advancing the tissue anchor distally with respect to the segments in a manner that pushes the segments, such that the segments pivot laterally away from each other.

Example 61. The method according to example 60, wherein the tissue anchor includes a head portion having a sloped surface that is dimensioned such that the advancement of the tissue anchor distally with respect to the segments slides the sloped surface along the segments in a manner that pushes the segments to pivot laterally away from each other.

Example 62. The method and/or system according to any of the above examples, wherein the tissue anchor is sterilized.

Example 63. The method and/or system according to any of the above examples, wherein the tube is sterilized.

Example 64. The system according to any of the above examples, wherein the driver is sterilized.

Example 65. The system according to any of the above examples, wherein the delivery tool is sterilized.

Although the operations of some of the disclosed examples are described in a particular, sequential order for convenient presentation, it should be understood that this manner of description encompasses rearrangement, unless a particular ordering is required by specific language set forth above. For example, operations or steps described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are discernible by one of ordinary skill in the art.

What is claimed:

1. A system for use with a tissue at an anatomical site of a subject, the system comprising:
a tissue anchor, including a tissue-engaging element and a driving interface which is fixed with respect to the tissue-engaging element; and
a delivery tool comprising:
a tube, having a blunt cap reversibly attached to and covering a distal end of the tube, the tube being transluminally advanceable in a distal direction toward the anatomical site; and
a driver that, while engaged with the driving interface of the tissue anchor, is configured to drive the tissue-engaging element through the blunt cap and into the tissue, such that the blunt cap becomes an anchor-head of the tissue anchor, secured to the tissue by the tissue-engaging element.

2. The system according to claim 1, wherein:
the tube defines a protrusion,
the blunt cap defines an indentation, and
the blunt cap is reversibly attached to the tube by the protrusion being disposed within the indentation.

3. The system according to claim 1, wherein:
the blunt cap defines a protrusion,
the tube defines an indentation, and
the blunt cap is reversibly attached to the tube by the protrusion being disposed within the indentation.

4. The system according to claim 1, wherein the driver is transluminally advanceable, within the tube, in the distal direction towards the anatomical site.

5. The system according to claim 1, wherein the tube is transluminally advanceable, in the distal direction towards the anatomical site, while the tissue anchor is disposed within the tube, proximally from the blunt cap.

6. The system according to claim 1, wherein the delivery tool further comprises a longitudinal catheter, and wherein the tube is disposed at a distal end of the longitudinal catheter.

7. The system according to claim 6, wherein the tube is attached to a distal end of the longitudinal catheter.

8. The system according to claim 1, wherein the blunt cap includes a predefined opening for passage of the tissue-engaging element of the tissue anchor through the blunt cap.

9. The system according to claim 1, wherein the blunt cap is pierceable by the tissue-engaging element during driving of the tissue-engaging element through the blunt cap of the tube.

10. The system according to claim 1, wherein:
the tissue-engaging element comprises a helical tissue-engaging element, and
the driver is configured to helically drive the helical tissue-engaging element through the blunt cap and into the tissue via application of torque to the driver.

11. The system according to claim 1, wherein the blunt cap is attached to the tube by snap fit engagement.

12. The system according to claim 11, wherein the snap fit engagement is configured such that driving the tissue-engaging element through the blunt cap and into the tissue facilitates detachment of the blunt cap from the tube.

13. The system according to claim 11, wherein the delivery tool is adapted to test anchoring of the tissue-engaging element to the tissue following driving of the tissue-engaging element through the blunt cap, via pulling of the tube such that (i) sufficient anchoring of the tissue-engaging element to the tissue results in the pulling of the tube overcoming a predefined strength of the snap fit engagement and thereby detaching the tube from the blunt cap, whereas (ii) insufficient anchoring of the tissue-engaging element to the tissue results in the pulling of the tube pulling the blunt cap and the tissue anchor away from the tissue.

14. The system according to claim 13, wherein the tissue-engaging element and the blunt cap are configured such that the driver can retract the tissue-engaging element proximally through the blunt cap.

15. A system for use with a subject, the system comprising:
a tissue anchor, including a tissue-engaging element and a driving interface which is fixed with respect to the tissue-engaging element; and
a delivery tool comprising:
a tube, having a blunt cap covering a distal end of the tube, and configured to be transluminally advanced toward an anatomical site of the subject with the tissue anchor disposed within the tube, proximally from the blunt cap; and
a driver, extending through the tube while engaged with the driving interface of the tissue anchor, and configured to advance the tissue-engaging element distally beyond the cap, and to anchor the tissue anchor at the anatomical site by driving the tissue-engaging element into tissue at the anatomical site.

16. The system according to claim 15, wherein the delivery tool further comprises a longitudinal catheter, and wherein the tube is at a distal end of the longitudinal catheter.

17. The system according to claim 15, wherein the blunt cap is integrally formed with the tube.

18. The system according to claim 15, wherein the blunt cap is attached to the distal end of the tube.

19. The system according to claim 18, wherein the tissue-engaging element of the tissue anchor is adapted to be driven through the blunt cap of the tube and into the tissue, such that the anchoring of the tissue anchor to the tissue secures the blunt cap against the tissue, thereby facilitating detachment of the blunt cap from the tube, and converting the blunt cap into an anchor-head of the tissue anchor.

20. A system for use with a subject, the system comprising:
a tissue anchor, including a tissue-engaging element and a driving interface which is fixed with respect to the tissue-engaging element;
a tube, having a blunt cap reversibly attached to and covering a distal end of the tube, the tube configured to be transluminally advanced toward an anatomical site of the subject with the tissue anchor disposed within the tube; and
a driver, extending through the tube, and configured to anchor the tissue anchor at the anatomical site by driving the tissue-engaging element into tissue at the anatomical site,
wherein the tissue-engaging element of the tissue anchor is adapted to be driven through the blunt cap of the tube and into the tissue, such that the anchoring of the tissue anchor to the tissue secures the blunt cap against the tissue, thereby facilitating detachment of the blunt cap from the tube, and converting the blunt cap into an anchor-head of the tissue anchor.
